(19) United States
(12) Patent Application Publication
(10) Pub. No.: US 2026/0066098 A1
(43) Pub. Date: Mar. 5, 2026

(54) SUPERIMPOSING IMAGES ANALYSIS SYSTEM FOR AVM

(71) Applicant: Imvitec Corporation, Taipei City (TW)

(72) Inventors: HERLEEYANDI MARKONI, Taipei City (TW); Wen-Cheng CHANG, Taipei City (TW); Jing-Ming GUO, Taipei City (TW); Hui-Ya HUNG, Taipei City (TW)

(21) Appl. No.: 19/000,126

(22) Filed: Dec. 23, 2024

(30) Foreign Application Priority Data
Sep. 5, 2024 (TW) 113133745

(51) Int. Cl. G16H 30/40; G06T 7/33; G06T 11/00

(52) U.S. Cl. CPC G16H 30/40; G06T 7/33; G06T 12/30; G06T 2207/30016; G06T 2210/62; G06T 2211/464

(57) ABSTRACT

A superimposing images analysis system for AVM includes an image capturing unit, an image superimposing and aligning unit, and a transparency adjustment unit. The image superimposing and aligning unit is connected to the image capturing unit, and the transparency adjustment unit is connected to the image superimposing and aligning unit. The image capturing unit captures a brain MRA image and a brain MRI image. The image superimposing and aligning unit receives the brain MRA image and brain MRI image from the image capturing unit, and performs superimposing and aligning with the received images to generate a superimposing brain image. The transparency adjustment unit adjusts the transparency of the brain MRA image or the brain MRI image in the superimposing brain image according to a transparency adjustment instruction so as to generate a compared superimposing brain image.

### CROSS REFERENCE TO RELATED APPLICATIONS

This Non-provisional application claims priority under 35 U.S.C. § 119(a) on Patent Application No(s). 113133745 filed in Taiwan, Republic of China on Sep. 5, 2024, the entire contents of which are hereby incorporated by reference.

### BACKGROUND

### Technology Field

The present disclosure relates to a superimposing images analysis system for AVM (arteriovenous malformations) and, in particular, to a superimposing images analysis system for analyzing superimposing images to detect out non-hemorrhagic AVM.

### Description of Related Art

Regarding medical examinations, doctors usually mark the AVM (arteriovenous malformation) region in the brain by simultaneously examining the T2 MRI image and the TOF MRA image obtained by TOF (time of flight) technology. In clinical practices, the aforementioned two images are obtained through different wave sequences and different imaging principles. Therefore, medical personnel often have to open different windows to view these images, and even have to use different image browsers to show these image files. In addition, since these images are presented separately, there is no consistency in alignment. In other words, the medical personnel must spend time in switching between the T2 MRI image and the TOF MRA image in order to compare the symptom information displayed by the two images.

In general, the TOF MRA image is mostly used to mark the ROI (region of interest) in the AVM region, while the T2 MRI image is commonly used in the identification of tissue segmentation in the AVM region. In other words, the two images are different in the presentations of symptom information and the uses of information.

Generally, doctors need to analyze the location of AVM and manually mark the AVM region. However, the marked AVM region usually contains the CSF (cerebrospinal fluid) region and the brain tissue region in addition to the lesion vessel region. Therefore, when making treatment plans, the medical personnel often need to use the T2 MRI image to compare the distributions of the lesion vessel region, the CSF region, and the brain tissue region in the ROI region, and then make the treatment plan based on the distributions thereof.

Furthermore, because the lesion vessel region is the critical area in the treatment, and the CSF and normal brain tissues have low tolerance to therapeutic doses. Therefore, the medical personnel need to carefully and manually dividing tissues, manually interpret the tissues and the relative positions between them, and manually analyze the therapeutic dose required for the lesions.

So far, before treatment planning for non-hemorrhagic AVM, medical personal have to spend a lot of time on marking and comparing different types of images. In addition, since the two images do not have actual alignment positions, it is easy to cause deviations in the actual positions, which may affect the quality of treatment. Therefore, it is desired to provide a system that allows medical personnel to use two different images to quickly and accurately determine the distributions of the lesion vessel region, the CSF region, and the brain tissue region in the ROI region, thereby providing a faster and higher-quality medical treatment to the patient.

### SUMMARY

An objective of this disclosure is to provide a superimposing images analysis system for AVM that can superimpose and align the MRA image and the MRI image so as to quickly and accurately determine the distributions of the lesion vessel region, the CSF region, and the brain tissue region in the ROI region.

To achieve the above, a superimposing images analysis system for AVM of this disclosure includes an image capturing unit, an image superimposing and aligning unit, and a transparency adjustment unit. The image superimposing and aligning unit is connected to the image capturing unit, and the transparency adjustment unit is connected to the image superimposing and aligning unit. The image capturing unit captures a brain MRA image and a brain MRI image. The image superimposing and aligning unit receives the brain MRA image and brain MRI image from the image capturing unit, and performs superimposing and aligning with the received images to generate a superimposing brain image. The transparency adjustment unit adjusts the transparency of the brain MRA image or the brain MRI image in the superimposing brain image according to a transparency adjustment instruction so as to generate a compared superimposing brain image (I_{O}).

In one embodiment, the superimposing images analysis system further includes an ROI marking unit connected to the image capturing unit, and the ROI marking unit performs ROI marking on an AVM region in the brain MRA image, thereby forming an ROI mark in the brain MRA image.

In one embodiment, the superimposing images analysis system further includes an ROI mark modifying unit connected to the image superimposing and aligning unit, and the ROI mark modifying unit modifies the ROI mark to form a modified ROI mark in the brain MRA image.

In one embodiment, the superimposing images analysis system further includes a tissue segmentation unit connected to the image capturing unit, and the tissue segmentation unit performs tissue segmentation marking on an AVM region in the brain MRI image, thereby forming a tissue segmentation mark in the brain MRI image.

In one embodiment, the superimposing images analysis system further includes a mask adjustment unit connected to the image superimposing and aligning unit. The mask adjustment unit adjusts a transparency of the ROI mark or a transparency of the tissue segmentation mark according to a mask adjustment instruction, so that the image superimposing and aligning unit generates an ROI superimposing brain image (I_{R}) or a tissue segmentation superimposing brain image (I_{S}).

In one embodiment, the transparency adjustment instruction contains an image transparency percentage of X %, and the transparency adjustment unit adjusts the transparency of the brain MRA image to X %, and adjusts the transparency of the brain MRI image to (100%−X %).

In one embodiment, the mask adjustment instruction contains a value Y of the transparency of the ROI mark or the tissue segmentation mark, and 0%≤Y≤100%.

In one embodiment, the brain MRA image is a TOF brain MRA image obtained by the TOF (time of flight) technology.

In one embodiment, the brain MRI image is a T2 brain MRI image.

In one embodiment, the tissue segmentation mark includes a lesion vessel region mark, a brain tissue region mark, or a CSF region mark.

In one embodiment, the superimposing images analysis system further includes a dose recommendation and displaying unit connected to the image superimposing and aligning unit. The dose recommendation and displaying unit generates a recommendation dose intensity based on a lesion vessel volume of the lesion vessel region mark, and displays the recommendation dose intensity on the compared superimposing brain image (I_{O}), the ROI superimposing brain image (I_{R}) or the tissue segmentation superimposing brain image (I_{S}).

As mentioned above, in the superimposing images analysis system of this disclosure, the image superimposing and aligning unit can quickly and accurately superimpose and align the TOF brain MRA image and the T2 brain MRI image, and the transparency adjustment unit can adjust the relative transparency of the brain MRA image and the brain MRI image in the superimposing brain image. Therefore, the medical personal can easily and quickly determine the distributions of the lesion vessel region, the CSF region, and the brain tissue region in the ROI region of the superimposing brain image. Moreover, the superimposing images analysis system further includes a dose recommendation and displaying unit for generating a recommendation dose intensity, and the recommendation dose intensity can be displayed on the compared superimposing brain image (I_{O}), the ROI superimposing brain image (I_{R}), or the tissue segmentation superimposing brain image (I_{S}) as a dose reference to the medical personal. Therefore, the medical personal can quickly and accurately make a proper treatment plan, thereby providing a faster and higher-quality medical treatment to the patient.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a schematic block diagram showing a superimposing images analysis system for AVM according to an embodiment of this disclosure;

FIG. 2A is a schematic diagram showing a superimposing brain image generated by superimposing and aligning a TOF brain MRA image and a T2 brain MRI image, wherein the transparency ratio thereof is 25%:75%;

FIG. 2B is a schematic diagram showing a superimposing brain image generated by superimposing and aligning a TOF brain MRA image and a T2 brain MRI image, wherein the transparency ratio thereof is 0%:100%;

FIG. 3 is a schematic block diagram showing a superimposing images analysis system for AVM according to another embodiment of this disclosure, wherein the superimposing images analysis system further includes an ROI marking unit and a mask adjustment unit;

FIG. 4A is a schematic diagram showing a TOF brain MRA image, wherein the transparency of the ROI mark is 75%;

FIG. 4B is a schematic diagram showing a TOF brain MRA image, wherein the transparency of the ROI mark is 25%;

FIG. 5 is a schematic block diagram showing a superimposing images analysis system for AVM according to another embodiment of this disclosure, wherein the superimposing images analysis system further includes an ROI mark modifying unit;

FIG. 6 is a schematic block diagram showing a superimposing images analysis system for AVM according to another embodiment of this disclosure, wherein the superimposing images analysis system further includes a tissue segmentation unit;

FIG. 7A is a schematic diagram showing a superimposing brain image generated by superimposing and aligning a TOF brain MRA image and a T2 brain MRI image containing a tissue segmentation mark;

FIG. 7B is a schematic diagram showing a superimposing brain image of FIG. 7A, wherein a transparency of the tissue segmentation mark in the T2 brain MRI image is 75%;

FIG. 7C is a schematic diagram showing a superimposing brain image of FIG. 7A, wherein a transparency of the tissue segmentation mark in the T2 brain MRI image is 25%;

FIG. 8 is a schematic block diagram showing a superimposing images analysis system for AVM according to another embodiment of this disclosure, wherein the superimposing images analysis system further includes a dose recommendation and displaying unit;

FIG. 9A is a schematic diagram showing a superimposing brain image generated by the superimposing images analysis system of FIG. 8, wherein the recommendation dose intensity is displayed in a transparency of 75% on the area of the ROI mark; and

FIG. 9B is a schematic diagram showing a superimposing brain image generated by the superimposing images analysis system of FIG. 8, wherein the recommendation dose intensity is displayed in a transparency of 25% on the area of the ROI mark.

### DETAILED DESCRIPTION OF THE DISCLOSURE

The present disclosure will be apparent from the following detailed description, which proceeds with reference to the accompanying drawings, wherein the same references relate to the same elements.

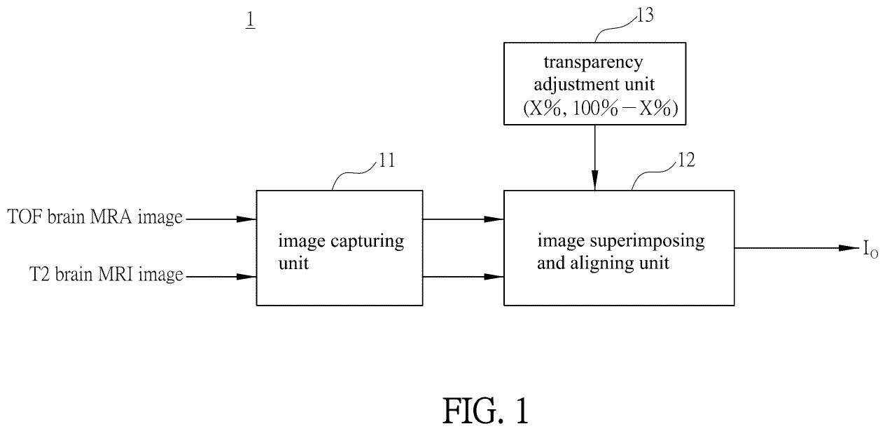

Referring to FIG. 1, a superimposing images analysis system 1 for AVM of this embodiment includes an image capturing unit 11, an image superimposing and aligning unit 12, and a transparency adjustment unit 13. The image capturing unit 11 and the transparency adjustment unit 13 are individually connected to the image superimposing and aligning unit 12. To be noted, these units can be connected in communication connection or electrical connection, and this disclosure is not limited thereto.

As shown in FIG. 1, the image capturing unit 11 can separately capture a brain MRA (Magnetic Resonance Angiography) image and a brain MRI (Magnetic Resonance Imaging) image. For example, the brain MRA image is a TOF MRA image generated using time-of-flight (TOF) technology, and the brain MRI image can be a T2 brain MRI image (hereinafter also referred to as T2 image, or MRI image). To be noted, the image capturing unit 11 may further include a storage to store the TOF brain MRA images and the T2 brain MRI images. The image superimposing and aligning unit 12 receives the TOF brain MRA image and the T2 brain MRI image from the image capturing unit 11, and performs superimposing and aligning with the received images. Specifically, the image superimposing and aligning unit 12 automatically superimposes and aligns the spatial domains and the slice areas in the TOF brain MRA image and the T2 brain MRI image, and may further align the common reference points (not shown) in the TOF brain MRA image and the T2 brain MRI image, thereby finishing the superimposing and aligning processes of the TOF brain MRA image and the T2 brain MRI image to generate a superimposing brain image.

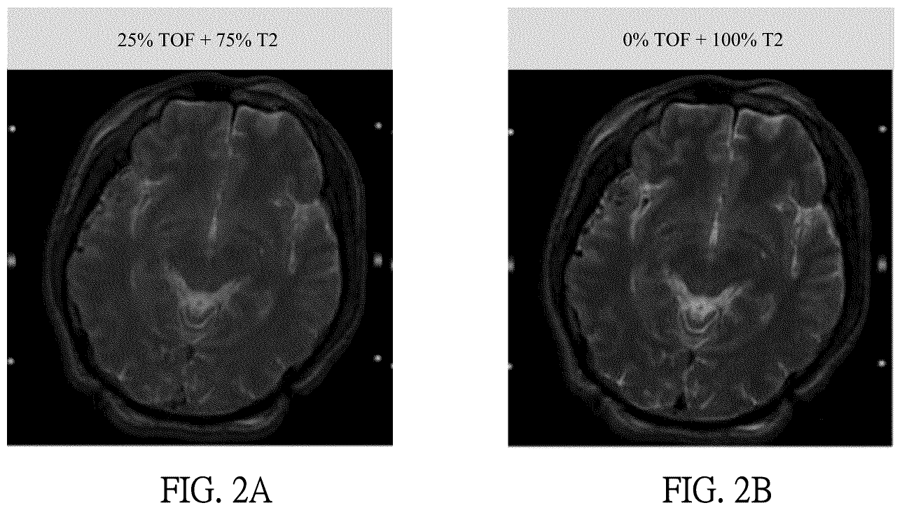

Referring to FIGS. 2A and 2B, the transparency adjustment unit 13 adjusts the relative transparencies of the TOF brain MRA image and the T2 brain MRI image in the superimposing brain image according to a transparency adjustment instruction. In brief, the transparency adjustment unit 13 can adjust the transparency percentage of the TOF brain MRA image or the T2 brain MRI image in the superimposing brain image according to the transparency adjustment instruction set by the system user. For example, when the user adjusts the transparency percentage of the TOF brain MRA image to X %, the transparency percentage of the T2 brain MRI image can be automatically adjusted to (100%−X %), so that the image superimposing and aligning unit 12 can generate and output a compared superimposing brain image (I_{O}). As shown in FIG. 2A, it represents a compared superimposing brain image (I_{O}), wherein the transparency ratio of the superimposed TOF brain MRA image and T2 brain MRI image is 25%:75%. In another case, as shown in FIG. 2B, it represents another compared superimposing brain image (I_{O}), wherein the transparency ratio of the superimposed TOF brain MRA image and T2 brain MRI image is 0%:100%.

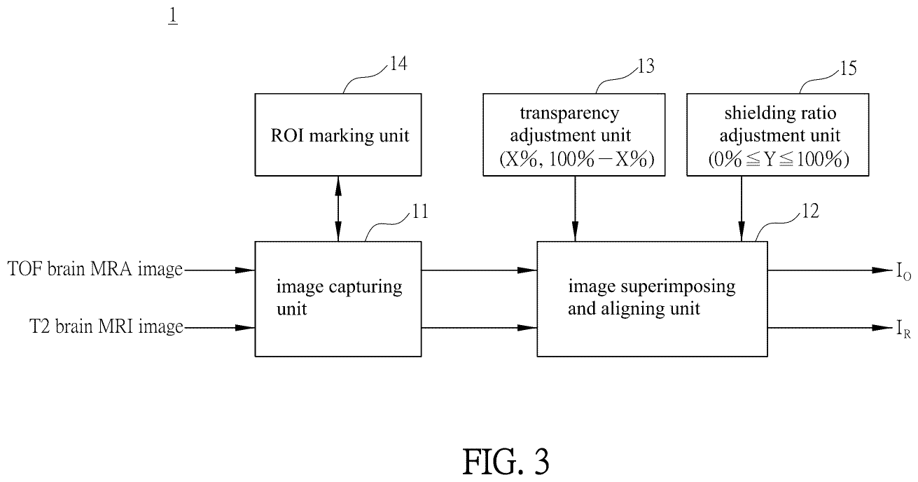

Referring to FIG. 3, the superimposing images analysis system 1 may further include an ROI marking unit 14 and a mask adjustment unit 15. The ROI marking unit 14 is connected to the image capturing unit 11, and the ROI marking unit 14 performs ROI marking on a non-hemorrhagic AVM region in the TOF brain MRA image received from the image capturing unit 11, thereby forming an ROI mark 141 in the TOF brain MRA image. To be noted, in this embodiment, the ROI mark 141 can be formed by manually marking or automatically marking with AI model.

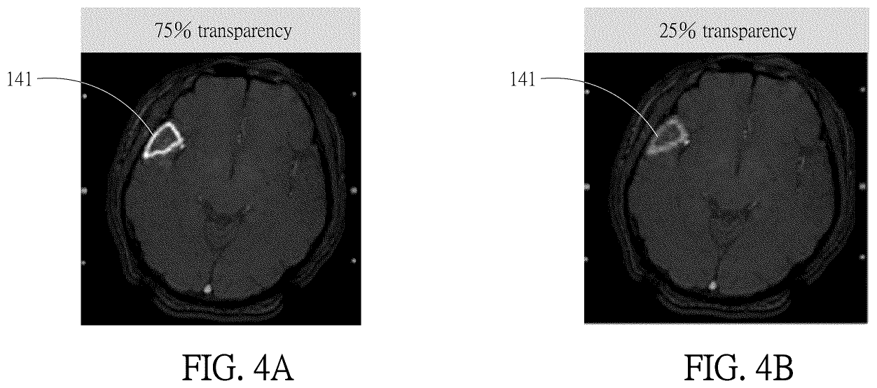

Referring to FIGS. 4A and 4B, the mask adjustment unit 15 can adjusts a transparency percentage Y (or the opacity of mask) of the ROI mark 141 in the TOF brain MRA image of the superimposing brain image according to a mask adjustment instruction. In this embodiment, the transparency percentage Y (or the opacity of mask) can be adjusted within a range from 0% to 100%. In brief, the user can adjust the transparency percentage of the ROI mark 141 through the mask adjustment unit 15, so that the image superimposing and aligning unit 12 can generate an ROI superimposing brain image (I_{R}). FIG. 4A is a schematic diagram showing an ROI superimposing brain image (I_{R}), wherein the transparency of the ROI mark 141 in the TOF brain MRA image is 75%. FIG. 4B is a schematic diagram showing another ROI superimposing brain image (I_{R}), wherein the transparency of the ROI mark 141 in the TOF brain MRA image is 25%.

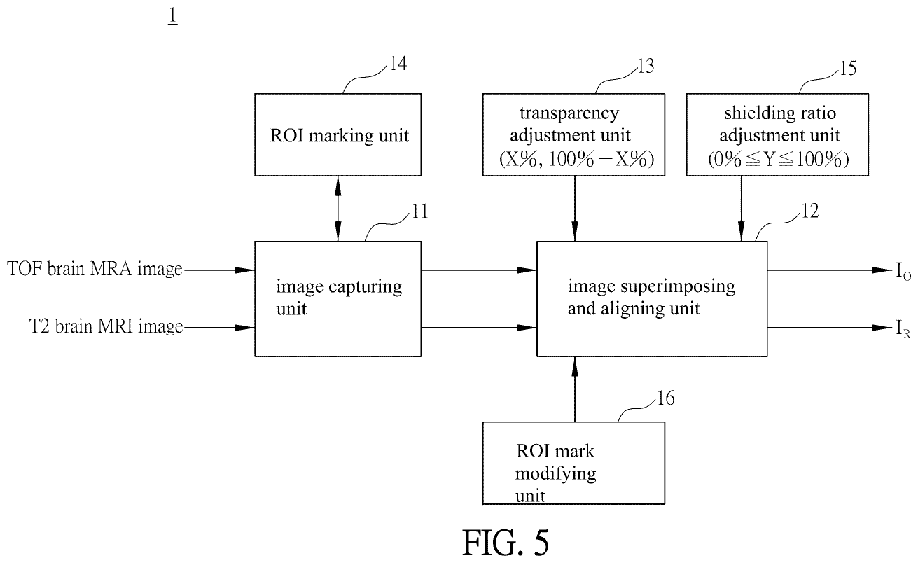

Referring to FIG. 5, the superimposing images analysis system 1 may further include an ROI mark modifying unit 16 connected to the image superimposing and aligning unit 12. The user may utilize the ROI mark modifying unit 16 to manually modify the ROI mark 141 to form a modified ROI mark in the brain MRA image.

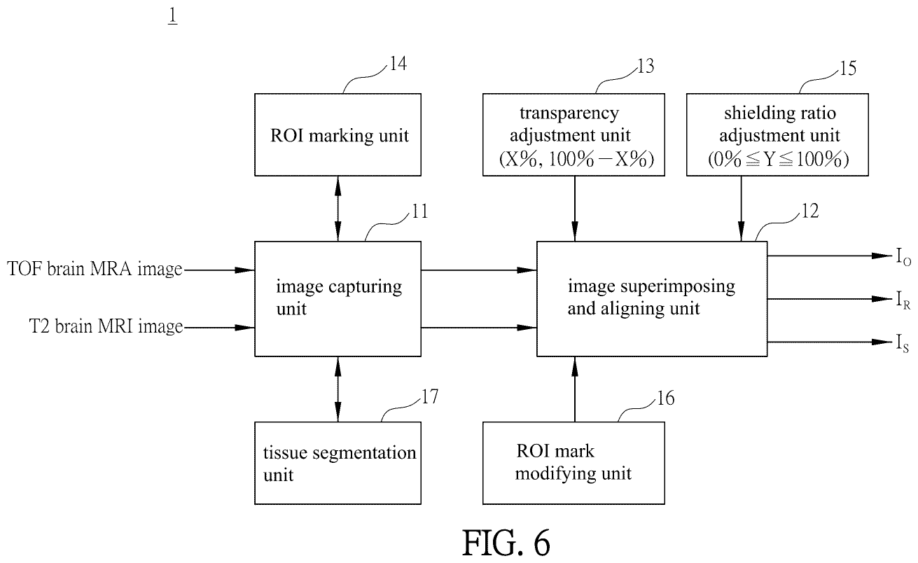

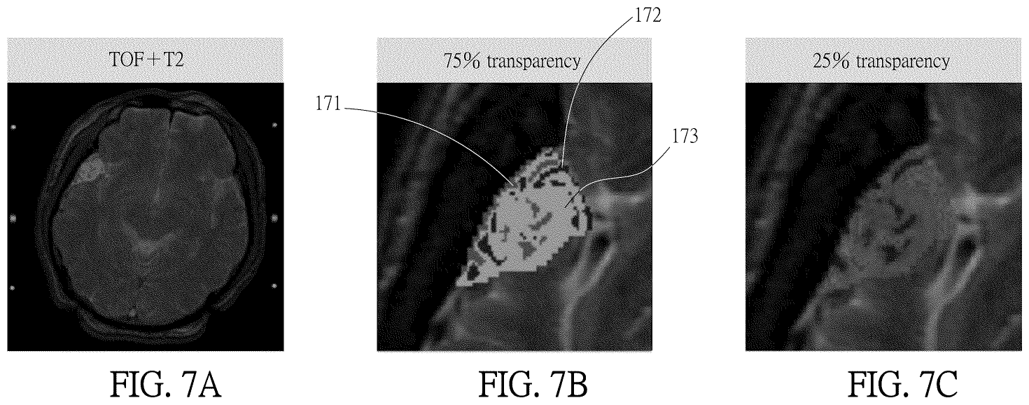

As shown in FIG. 6, the superimposing images analysis system 1 may further include a tissue segmentation unit 17 connected to the image capturing unit 11, and the tissue segmentation unit 17 performs tissue segmentation marking on the non-hemorrhagic AVM region in the T2 brain MRI image received from the image capturing unit 11. As shown in FIG. 7B, the tissue segmentation mark generally includes a lesion vessel region mark 171, a brain tissue region mark 172, and/or a CSF region mark 173. To be noted, in this embodiment, the tissue segmentation mark can be formed by manually marking or automatically marking with AI model. To be noted, the process of tissue segmentation marking can also be applied to the modified ROI mark in the brain MRA image, and this disclosure is not limited.

As mentioned above, in this embodiment, the user may utilize the mask adjustment unit 15 to adjust the transparency percentage Y (or the opacity of mask) of the tissue segmentation mark in the T2 brain MRI image of the superimposing brain image according to a mask adjustment instruction. In this embodiment, the transparency percentage Y (or the opacity of mask) can be adjusted within a range from 0% to 100%. In brief, the user can adjust the transparency percentage of the tissue segmentation mark through the mask adjustment unit 15, so that the image superimposing and aligning unit 12 can generate a tissue segmentation superimposing brain image (I_{S}). FIG. 7B is a schematic diagram showing a tissue segmentation superimposing brain image (I_{S}), wherein the transparency of the tissue segmentation mark in the T2 brain MRI image is 75%. FIG. 7C is a schematic diagram showing another tissue segmentation superimposing brain image (I_{S}), wherein the transparency of the tissue segmentation mark in the T2 brain MRI image is 25%.

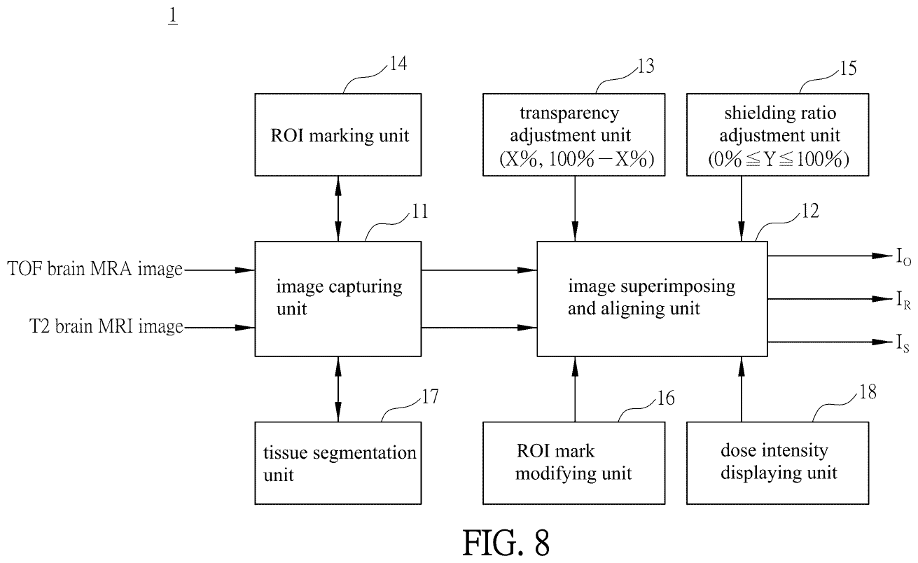

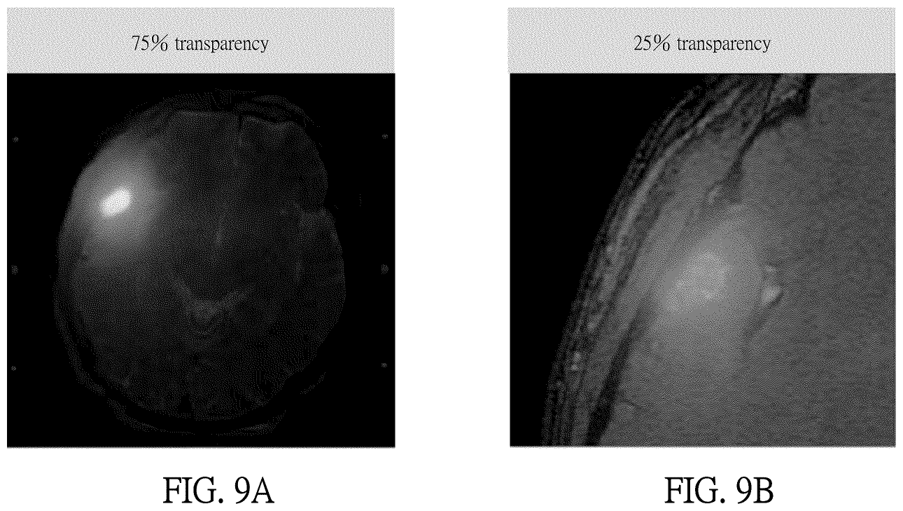

With reference to FIG. 8, the superimposing images analysis system 1 of this embodiment may further include a dose recommendation and displaying unit 18 connected to the image superimposing and aligning unit 12. The dose recommendation and displaying unit 18 generates a recommendation dose intensity for the treatment reference based on a lesion vessel volume of the lesion vessel region mark 171, and displays the recommendation dose intensity on the compared superimposing brain image (I_{O}), the ROI superimposing brain image (I_{R}) or the tissue segmentation superimposing brain image (I_{S}). Accordingly, the medical personal can catch the recommendation dose intensity while analyzing any of the above-mentioned brain images. In this embodiment, the recommendation dose intensity refers to the radiation dose intensity that medical personnel deliver when performing radiotherapy. Therefore, the medical personnel can easily predict the actual treatment situation in advance through the dose recommendation and displaying unit 18, thereby allowing the medical personnel to quickly and conveniently make the treatment plan. For example, as shown in FIG. 9A, the system user (medical personnel) can utilize the dose recommendation and displaying unit 18 to display the recommendation dose intensity in a transparency of 75% on the area of the ROI mark in the ROI superimposing brain image (I_{R}). In another case, as shown in FIG. 9B, the system user (medical personnel) can utilize the dose recommendation and displaying unit 18 to display the recommendation dose intensity in a transparency of 25% on the area of the ROI mark in the ROI superimposing brain image (I_{R}).

In summary, in the superimposing images analysis system 1 of this disclosure, the image superimposing and aligning unit 12 can quickly and accurately superimpose and align the TOF brain MRA image and the T2 brain MRI image, and the transparency adjustment unit 13 can adjust the relative transparency of the brain MRA image and the brain MRI image in the superimposing brain image. Therefore, the medical personal can easily and quickly determine the distributions of the lesion vessel region, the CSF region, and the brain tissue region in the ROI region of the superimposing brain image. Moreover, the superimposing images analysis system 1 further includes a dose recommendation and displaying unit 18 for generating a recommendation dose intensity, which can be displayed on the compared superimposing brain image (I_{O}), the ROI superimposing brain image (I_{R}), or the tissue segmentation superimposing brain image (I_{S}) as a dose reference to the medical personal. Therefore, the medical personal can quickly and accurately make a proper treatment plan, thereby providing a faster and higher-quality medical treatment to the patient.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A superimposing images analysis system for AVM (arteriovenous malformation), comprising:
an image capturing unit capturing a brain MRA (Magnetic Resonance Angiography) image and a brain MRI (Magnetic Resonance Imaging) image;
an image superimposing and aligning unit connected to the image capturing unit, wherein the image superimposing and aligning unit receives the brain MRA image and brain MRI image from the image capturing unit, and performs superimposing and aligning with the received images to generate a superimposing brain image; and
a transparency adjustment unit connected to the image superimposing and aligning unit, wherein the transparency adjustment unit adjusts a transparency of the brain MRA image or the brain MRI image in the superimposing brain image according to a transparency adjustment instruction so as to generate a compared superimposing brain image.

2. The superimposing images analysis system of claim 1, further comprising an ROI (Region of Interest) marking unit connected to the image capturing unit, wherein the ROI marking unit performs ROI marking on an AVM region in the brain MRA image, thereby forming an ROI mark in the brain MRA image.

3. The superimposing images analysis system of claim 2, further comprising an ROI mark modifying unit connected to the image superimposing and aligning unit, wherein the ROI mark modifying unit modifies the ROI mark.

4. The superimposing images analysis system of claim 2, further comprising a mask adjustment unit connected to the image superimposing and aligning unit, wherein the mask adjustment unit adjusts a transparency of the ROI mark according to a mask adjustment instruction, so that the image superimposing and aligning unit generates an ROI superimposing brain image.

5. The superimposing images analysis system of claim 4, wherein the mask adjustment instruction contains a value Y of the transparency of the ROI mark, and 0%≤Y≤100%.

6. The superimposing images analysis system of claim 1, further comprising a tissue segmentation unit connected to the image capturing unit, wherein the tissue segmentation unit performs tissue segmentation marking on an AVM region in the brain MRI image, thereby forming a tissue segmentation mark in the brain MRI image.

7. The superimposing images analysis system of claim 6, further comprising a mask adjustment unit connected to the image superimposing and aligning unit, wherein the mask adjustment unit adjusts a transparency of the tissue segmentation mark, so that the image superimposing and aligning unit generates a tissue segmentation superimposing brain image.

8. The superimposing images analysis system of claim 7, wherein the mask adjustment instruction contains a value Y of the transparency of the tissue segmentation mark, and 0%≤Y≤100%.

9. The superimposing images analysis system of claim 6, wherein the tissue segmentation mark comprises a lesion vessel region mark, a brain tissue region mark, or a CSF (cerebrospinal fluid) region mark.

10. The superimposing images analysis system of claim 6, further comprising:
a dose recommendation and displaying unit connected to the image superimposing and aligning unit, wherein the dose recommendation and displaying unit generates a recommendation dose intensity based on a lesion vessel volume of the lesion vessel region mark, and displays the recommendation dose intensity on the compared superimposing brain image, or the tissue segmentation superimposing brain image.

11. The superimposing images analysis system of claim 1, wherein the transparency adjustment instruction contains an image transparency percentage of X %, and the transparency adjustment unit adjusts the transparency of the brain MRA image to X %, and adjusts the transparency of the brain MRI image to (100%−X %).

12. The superimposing images analysis system of claim 1, wherein the brain MRA image is a TOF brain MRA image obtained by a TOF (time of flight) technology.

13. The superimposing images analysis system of claim 1, wherein the brain MRI image is a T2 brain MRI image.
